(19) United States
(12) Patent Application Publication
(10) Pub. No.: US 2021/0100965 A1
(43) Pub. Date: Apr. 8, 2021

(54) INHALER WITH SYNTHETIC JETTING

(71) Applicant: MICRODOSE THERAPEUTX, INC., Ewing, NJ (US)

(72) Inventor: Robert LEONARD, Jamison, PA (US)

(73) Assignee: MICRODOSE THERAPEUTX, INC., Ewing, NJ (US)

(21) Appl. No.: 16/500,229

(22) PCT Filed: May 22, 2018
(86) PCT No.: PCT/US2018/033829

§ 371 Date: Oct. 2, 2019

Related U.S. Application Data
(60) Provisional application No. 62/511,778, filed on May 26, 2017.

(51) Int. Cl. A61M 15/00

(52) U.S. Cl. CPC A61M 15/001; A61M 15/0086; A61M 15/0021; A61M 2202/0078; A61M 2205/502; A61M 2205/8206; A61M 2202/064; A61M 15/0091

(57) ABSTRACT

A dry powder inhaler consisting of a reusable base unit and a disposable drug package is disclosed. The reusable portion may house a transducer, a controller, battery and user interface. The disposable portion may house a dose pellet in a sealed dose chamber that includes an integrated mouthpiece. A user may couple the disposable portion to the reusable portion of the inhaler. The inhaler may sense the user's breathe and synchronize delivery of the pharmaceutical or drug to the user.

### CROSS-REFERENCE TO PRIOR APPLICATIONS

This application claims priority to U.S. Provisional Patent Application No. 62/511,778, filed May 26, 2017, which is hereby expressly incorporated by reference in its entirety.

### FIELD

The embodiments relate generally to the field of delivery of pharmaceuticals and drugs. Particular utility may be found in the delivery of a pharmaceutical or drug to a patient utilizing a portable reusable base unit and a disposable drug package and will be described in connection with such utility, although other utilities are contemplated.

### BACKGROUND

Certain diseases of the respiratory tract are known to respond to treatment by the direct application of therapeutic agents. As these agents are most readily available in dry powdered form, their application is most conveniently accomplished by inhaling the powdered material through the nose or mouth. This powdered form results in the better utilization of the medication in that the drug is deposited exactly at the site desired and where its action may be required; hence, very minute doses of the drug are often equally as efficacious as larger doses administered by other means, with a consequent marked reduction in the incidence of undesired side effects and medication cost. Alternatively, the drug in powdered form may be used for treatment of diseases other than those of the respiratory system. When the drug is deposited on the very large surface areas of the lungs, it may be very rapidly absorbed into the blood stream; hence, this method of application may take the place of administration by injection, tablet, or other conventional means.

Existing dry powder inhalers (DPIs) usually have a means for introducing a drug (active drug plus carrier) into a high velocity air-stream. The high velocity air-stream is used as the primary mechanism for breaking up the cluster of micronized particles or separating the drug particles from the carrier. These existing devices present several problems and possess several disadvantages. First, conventional DPIs, generally being passive devices, require the user to forcefully exhale then deeply inhale for optimal drug delivery. Such a disadvantage impacts more severely affected patients by requiring them to sustain difficult breathing patterns through an inhaler with a large amount of resistance. A need exists for a device which enables the user to breathe normally during dosing.

Further, conventional DPIs are highly sophisticated devices which are suited for consumers in highly developed nations. There is a market need for a simplified design that is more cost competitive for developing nations. Such a device may address many of the challenges of conventional DPIs such as eliminating the complicated dose advance mechanisms, simplifying the human factors design, and reducing the product cost. For example, many conventional multi-dose inhalers utilize blister strips or a series of individual blisters which require complicated mechanisms for reliable dose advance. Moreover, a sophisticated multi-dose inhaler may include an electric motor coupled to software for controlling the dose advance within a cartridge. These multi-dose inhalers may also provide wireless connectivity and a LCD user interface. Storage for the multiple dosages also enlarges the size of the disposable drug cartridge. All these factors result in an inhaler that may be too expensive for developing markets thus denying the unique drug delivery technology to those who need it most.

### SUMMARY

Embodiments described herein relate to methods, apparatuses, and/or systems for delivering a dose of a pharmaceutical or drug through an inhaler. In certain embodiments, the inhaler may comprise a reusable base unit and a disposable drug package. In some embodiments, the reusable portion may house a transducer, a controller, battery and user interface. In other embodiments, the disposable portion may house a dose of medicament in a sealed dose chamber that includes an integrated mouthpiece. In one embodiment, during operation, a user may insert the disposable portion onto the reusable portion of the inhaler. Next, the user may remove a seal attached to the disposable portion to expose a pharmaceutical or drug located within the dose chamber. The user may then bring the mouthpiece to their lips and start to breathe normally. During this time, the inhaler may sense the user's breathe and synchronize delivery of the pharmaceutical or drug to the user utilizing synthetic jetting. During use, an indicator, such as the light, at the distal end of the reusable portion may illuminate to indicate proper function of the inhaler.

In another embodiment, the transducer of the reusable portion of the inhaler may create an acoustic wave that aerosolizes the dry powder pharmaceutical or drug located in the dosing chamber via synthetic jetting. The aerosolized medication may be emitted into the airflow flow conduit and is entrained into the inhaled air via the mouthpiece and thereby into the user. Inhalation may be actively detected by the inhaler to synchronize delivery of the pharmaceutical or drug to the user. The pharmaceutical or drug may be prepared by compressing the micronized dry powder into a single pre-metered dose pellet which is packaged into individual blister packs. In one embodiment, a single pre-metered dose pellet may include a container closure system and an acoustic chamber for synthetic jetting.

In accordance with one embodiment, a dry powder inhaler is provided. The dry powder inhaler includes a first portion including a dry powder medicament, a dosing chamber configured to receive the medicament, and a mouthpiece configured to deliver the medicament in aerosolized form to the user. The dry powder inhaler also includes a second portion including a transducer configured to aerosolize the medicament when the transducer is activated and a controller configured to activate the transducer in response to an activation event. The first portion and the second portion may be coupled together at a connection point. In some embodiments, the first portion and the second portion include outer tubular housings extending in a longitudinal direction. In some embodiments, the inhaler is about 5-15 millimeters in diameter and about 80-150 millimeters in length when the first portion and the second portion are coupled together. In some embodiments, the first portion is disposable and the second portion is reusable.

In accordance with another embodiment, a method for delivering a dose of a drug with an inhaler is provided. The method includes coupling a first and second portion of the inhaler, providing a dry powder medicament located in the first portion of the inhaler, and aerosolizing the dry powder medicament via a transducer in the second portion of the inhaler. The transducer may be activated in response to an activation event via controller in the second portion of the inhaler. The method further includes receiving an aerosolized form of the dry powder in a dosing chamber within the first portion of the inhaler and delivering the aerosolized dry powder through a mouthpiece of the first portion of the inhaler. In some embodiments, the first portion and the second portion include outer tubular housings extending in a longitudinal direction. In some embodiments, the inhaler is about 5-15 millimeters in diameter and about 80-150 millimeters in length when the first portion and the second portion are coupled together. In some embodiments, the first portion is disposable and the second portion is reusable.

These methods, apparatuses, and/or systems provide significant advantages. First, the inhaler provides a simplified design eliminating the complicated dose advance mechanisms which may be more cost competitive in the market. Further, the synthetic jetting provided by the inhaler enables the user to breathe normally during dosing as compared to conventional passive inhalers which require the user to forcefully exhale then deeply inhale for optimal drug delivery.

Various other aspects, features, and advantages will be apparent through the detailed description and the drawings attached hereto. It is also to be understood that both the foregoing general description and the following detailed description are exemplary and not restrictive of the scope of the embodiments. As used in the specification and in the claims, the singular forms of “a”, “an”, and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

FIGS. 1A-C show a perspective view of an inhaler, in accordance with one or more embodiments.

FIGS. 2A and B show a perspective view of the coupling and initiation of operation of an inhaler, in accordance with one or more embodiments.

FIGS. 3A and B show a zoomed perspective view of an inhaler, in accordance with one or more embodiments.

FIG. 4 shows a flowchart of a method of delivering a dose of a drug with an inhaler, in accordance with one or more embodiments.

### DETAILED DESCRIPTION

In the following description, for the purposes of explanation, numerous specific details are set forth in order to provide a thorough understanding of the embodiments. It will be appreciated, however, by those having skill in the art that the embodiments may be practiced without these specific details or with an equivalent arrangement. In other instances, well-known structures and devices are shown in block diagram form in order to avoid unnecessarily obscuring the embodiments of the invention.

The present embodiments relate to a device for administering medicament as a dry powder for inhalation by a user. Some embodiments of the device may be classified as a dry powder inhaler (DPI). Some embodiments of the device may also be classified as a dry powder nebulizer (as opposed to a liquid nebulizer), particularly when tidal breathing is used to deliver dry powder medicament over one or more inhalations. The device may be referred to herein interchangeably as a “device” or an “inhaler,” both of which refer to a device for administering medicament as a dry powder for inhalation by a subject, and most preferably when tidal breathing is used. “Tidal breathing” preferably refers to inhalation and exhalation during normal breathing at rest, as opposed to forceful breathing.

Structure of an Inhalation Device

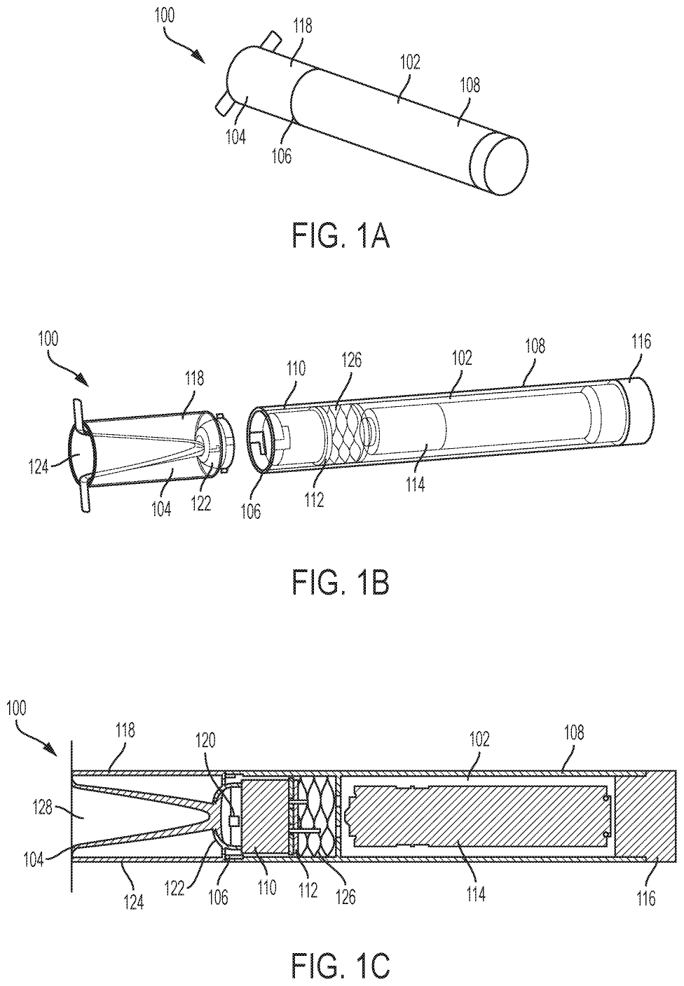

FIGS. 1A-C show an inhaler 100 configured to receive a user's inhale through the mouthpiece of the device, preferably via tidal breathing, and deliver a dose of medicament over one or more consecutive inhalations. As shown in FIGS. 1A-C, inhaler 100 may include a reusable portion 102 and a disposable portion 104, which may be coupled together at a connection point 106 or by other convenience such as a snug-fit, detent, clamp and/or clasp. In one embodiment, the reusable portion 102 may include an outer tubular housing 108 extending in a longitudinal direction for housing a transducer 110, controller 112, battery 114 and user interface 116. The disposable portion 104 may also include an outer tubular housing 118 extending in a longitudinal direction for housing a medicament dose 120 in a sealed dose chamber 122 that may include an integrated mouthpiece 124. In one embodiment, when the outer tubular housings 108, 118 of the reusable portion 102 and the disposable portion 104 are coupled together, the inhaler may have dimensions comparable to an electronic cigarette (5-15 mm in diameter and 80-150 mm in length). In another embodiment, the outer tubular housing can be a single tube housing both the reusable portion 102 and a disposable portion 104 and the entire inhaler can be disposable. It should be appreciated that the size and shape of inhaler housing 100 may vary to accommodate various aforementioned components.

In one embodiment, with respect to connection point 106, a user interface 116 (e.g. LED) may be arranged as an endcap of a distal end of the outer tubular housing 108 of the reusable portion 102. The user interface 116 may be electronically connected to the battery 114 via controller 112 of the inhaler 100. In some embodiments, user interface 116 may provide an indication that proper function of the inhaler has occurred as will be described in greater detail below. In another embodiment, with respect to connection point 106, transducer 110 may be arranged as the endcap of the proximal end of the outer tubular housing 108 of the reusable portion 102. Transducer 110 may also be electronically connected to the battery 114 controller 112 of the inhaler. In some embodiment, as will be described in greater detail below. A conductive spring 126 and the controller 112 may be arranged between transducer 110 and battery 114 to ensure a secure electrical and physical connection between various aforementioned components.

In some embodiments, with respect to connection point 106, the medicament dose 120 and dose chamber 122 may be arranged at the proximal end of the outer tubular housing 118 of the disposable portion 104 such that when the reusable portion 102 and a disposable portion 104 are coupled together transducer 100 may vibrate to aerosolize and transfer the medicament dose 120 into the dosing chamber 122. In some embodiments, with respect to connection point 106, mouthpiece 124 is located at the distal end of the outer tubular housing 118 of the disposable portion 104 such that the user may receive delivery of the pharmaceutical or drug from the synthetic jetting provided by transducer 100 and dosing chamber 122. In some embodiments, a thin membrane may be sealed to the bottom of the dose chamber 122 to ensure a secure connection to transducer 100 when the reusable portion 102 and the disposable portion 104 are coupled together. In some embodiments, an air flow conduit 128 may be arranged between the dosing chamber 122 and mouthpiece 124 and configured to allow air to travel through the inhaler 100 when a user inhales through a mouthpiece 124.

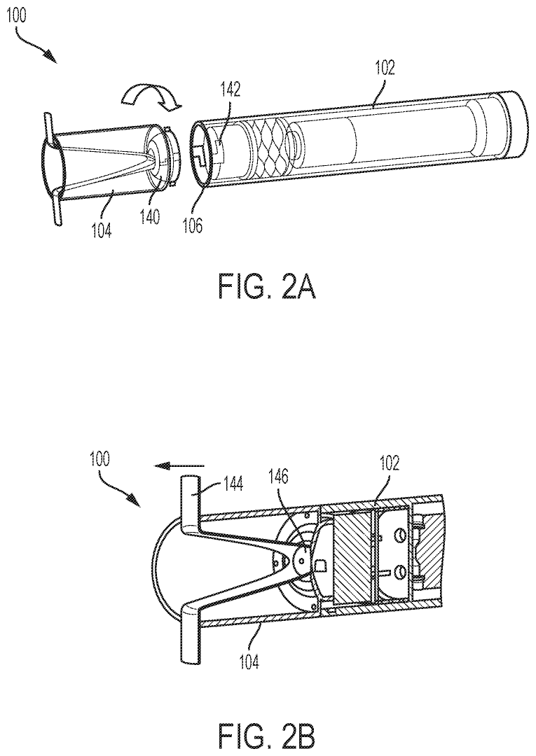

With respect to FIGS. 2A and B, the coupling and initiation of operation of inhaler 100 is illustrated. As shown in FIG. 2A, inhaler 100 may include a reusable portion 102 and a disposable portion 104, which may be coupled together at a connection point 106 or by other convenience such as a snug-fit, detent, clamp and/or clasp. For example, disposable portion 104 may include one or more guides 140 which fit into slots 142 of reusable portion 102 which may be secured via a twisting motion. In some embodiments, conductive spring 126 may provide resistance during the coupling of reusable portion 102 and a disposable portion 104 to provide a secure connection. With reference to FIG. 2B, after coupling of the reusable portion 102 and the disposable portion 104 is complete, the user may pull on tabs 144 to remove a seal 146 to expose the dose chamber 122 during a first use. For example, tabs 144 may be connected to the seal 146 located at the opposite end of disposable portion 104 by one or more longitudinal members configured to assist in removal of the seal from the dosing chamber 122. After removal of the seal 146 of the dosing chamber 122, the user may then bring the mouthpiece 124 to their lips and start to breathe normally. During this time, the inhaler may sense the user's breathe and synchronize delivery of the pharmaceutical or drug to the user utilizing synthetic jetting as described in greater detail below. Other embodiments may use a rubber plug to seal the dose chamber or mechanisms to uncover the holes in the dose chamber.

Operation of an Inhalation Device

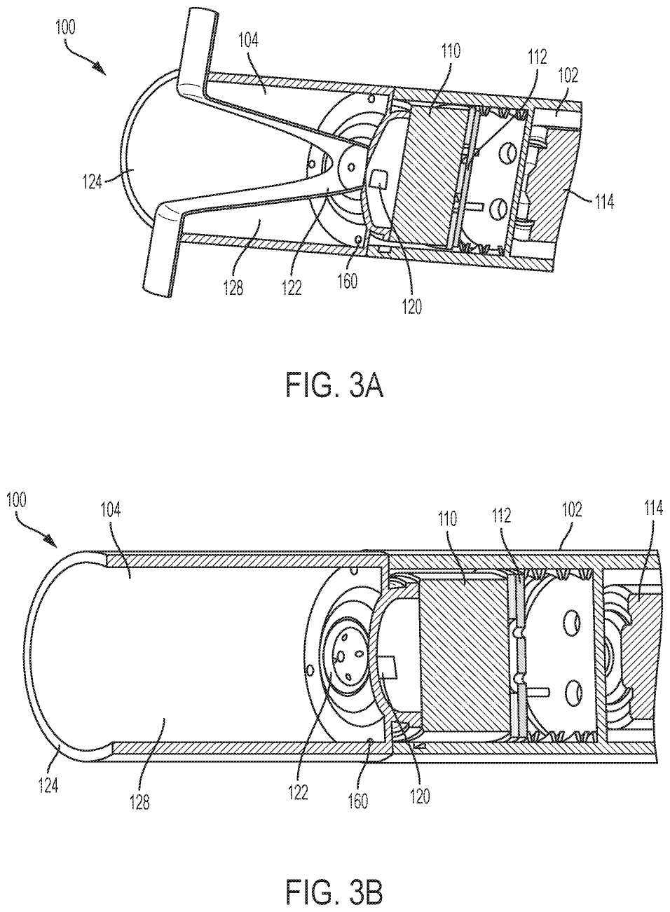

In one embodiment illustrated in FIGS. 3A and B, the inhaler 100 may be configured to activate transducer 110 to deliver a complete medicament dose 120 to a user via synthetic jetting. During operation, when the user inhales through the mouthpiece 124, air is drawn into the inhaler's air flow conduit 128 via air vents 160, and out of the mouthpiece 124 into the user's lungs; as air is being inhaled through the air flow conduit 128, dry powder medicament is expelled into the air flow conduit 128 and becomes entrained in the user's inhaled air. Thus, the air flow conduit 128 preferably defines an air path from the air vents 160 to the outlet (i.e., the opening that is formed by the mouthpiece). Each breath cycle includes an inhalation and an exhalation, i.e., each inhalation is followed by an exhalation, so consecutive inhalations preferably refer to the inhalations in consecutive breath cycles. After each inhalation, the user may exhale outside of the inhaler (e.g., by removing his or her mouth from the mouthpiece and expelling the inhaled air off to the side). In one embodiment, consecutive inhalations refer to each time a user inhales through the inhaler which may or may not be each time a patient inhales their breath.

In one embodiment, the inhaler 100 may contain a single pre-metered dose 120 of a dry powder drug composition comprising at least one medicament. As used herein, the pre-metered dose 120 may include a container that is suitable for containing a dose of dry powder medicament. According to a preferred embodiment, the pre-metered dose 120 may be arranged within the disposable portion 104 of inhaler 100, which comprises a base sheet in which pre-metered dose 120 is formed to define pockets therein for containing distinct medicament doses and a dose chamber 122 which is sealed in such a manner that the seal 146 of the dose chamber 122 can be peeled there by providing access to the medicament of the pre-metered dose 120.

In some embodiments, inhaler 100 may be configured to activate the transducer 110 one or more times to deliver a complete pharmaceutical dose from a dose pellet 120 and dose chamber 122 to a user. In one embodiment, the inhaler 100 may include an air flow conduit 128 configured to allow air to travel through the inhaler 100 when a user inhales through a mouthpiece 124. For example, the controller 114 may be configured to activate a transducer 102 when an activation event is detected. In some embodiments, the activation event may be the removal of the seal 146 from the dose chamber 122. In other embodiments, the inhaler 100 may include an inhalation sensor configured to detect airflow through the air flow conduit 124 and the activation event may be detection of an inhalation of the user. In another embodiment, the activation event may be a user inputted signal such as a push button located on the housing of the inhaler 100. The transducer 110 may be configured to vibrate, thereby vibrating the pharmaceutical, to aerosolize and transfer the pharmaceutical from the dose 120 into the dosing chamber 122. In one embodiment, the vibration of the transducer 102 also delivers the aerosolized pharmaceutical into the dosing chamber 118, through the air flow conduit 128, and to the user through mouthpiece 124. It should be appreciated that the delivery of the pharmaceutical to the user is accomplished via synthetic jetting.

The transducer 110 may be a piezoelectric element made of a material that has a high-frequency, and preferably, ultrasonic resonant vibratory frequency (e.g., about 15 to 50 kHz), and is caused to vibrate with a particular frequency and amplitude depending upon the frequency and/or amplitude of excitation electricity applied to the piezoelectric element. Examples of materials that can be used to comprise the piezoelectric element may include quartz and polycrystalline ceramic materials (e.g., barium titanate and lead zirconate titanate). Advantageously, by vibrating the piezoelectric element at ultrasonic frequencies, the noise associated with vibrating the piezoelectric element at lower (i.e., non-ultrasonic) frequencies can be avoided.

In some embodiments, the inhaler 100 may comprise an inhalation sensor that senses when a patient inhales through the device; for example, the sensor may be in the form of a pressure sensor, air stream velocity sensor or temperature sensor. According to one embodiment, an electronic signal may be transmitting to controller 112 contained in inhaler 100 each time the sensor detects an inhalation by a user such that the dose is delivered over several inhalations by the user. For example, the sensor may comprise a conventional flow sensor which generates electronic signals indicative of the flow and/or pressure of the air stream in the air flow conduit 128, and transmits those signals via electrical connection to controller 112 contained in inhaler 100 for controlling actuation of the transducer 110 based upon those signals. Preferably, sensor may be a pressure sensor. Non-limiting examples of pressure sensors that may be used in accordance with embodiments may include a microelectromechanical system (MEMS) pressure sensor or a nanoelectromechanical system (NEMS) pressure sensor herein. The inhalation sensor may be located in or near an air flow conduit 128 to detect when a user is inhaling through the mouthpiece 124.

Preferably, the controller 112 may be embodied as an application specific integrated circuit chip and/or some other type of very highly integrated circuit chip. Alternatively, controller 112 may take the form of a microprocessor, or discrete electrical and electronic components. As will be described more fully below, the controller 112 may control the power supplied from conventional power source 114 (e.g., a D.C. battery) to the transducer 110. The power may be supplied to the transducer 110 via electrical connection between the transducer 110 and the controller 112.

The memory may include non-transitory storage media that electronically stores information. The memory may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storage may store dosing technique, information determined by the processors, information received from sensor, or other information that enables the functionality as described herein.

During operation, controller 112 may also indicate proper function of inhaler 100 via user interface 116. For example, controller 112 may illuminate an LED 116 located at the end of inhaler 100 after delivery of the dose of the pharmaceutical or drug through the inhaler.

Exemplary Flowcharts

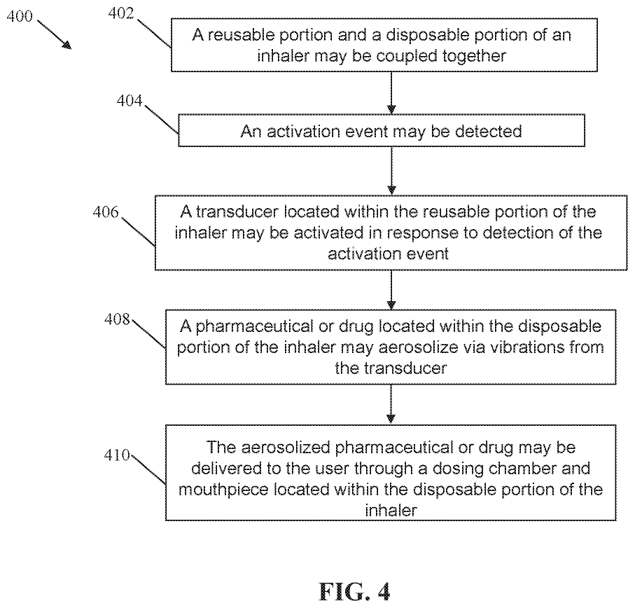

FIG. 4 illustrates a flowchart of an exemplary method 400 of delivering a dose of a drug with an inhaler, in accordance with one or more embodiments.

In an operation 402, a reusable portion and a disposable portion of an inhaler may be coupled together. In some embodiments, the reusable portion and the disposable portion may be coupled together at a connection point or by other convenience such as a snug-fit, detent, clamp and/or clasp. In some embodiments, the reusable portion may include an outer tubular housing extending in a longitudinal direction for housing a transducer, controller, battery and user interface. In some embodiments, the disposable portion may also include an outer tubular housing extending in a longitudinal direction for housing a medicament dose in a sealed dose chamber that may include an integrated mouthpiece.

In an operation 404, an activation event may be detected. In some embodiments, the activation event may be the removal of a seal from a dose chamber of the inhaler. In other embodiments, the activation event may be detection of an inhalation of the user. In other embodiments, the activation event may be a user inputted signal such as a push button located on the housing of the inhaler.

In an operation 406, a transducer located within the reusable portion of the inhaler may be activated in response to detection of the activation event. In some embodiment, the transducer may be configured to aerosolize the medicament when the transducer is activated. The transducer may be a piezoelectric element made of a material that has a high-frequency, and preferably, ultrasonic resonant vibratory frequency (e.g., about 15 to 50 kHz), and is caused to vibrate with a particular frequency and amplitude depending upon the frequency and/or amplitude of excitation electricity applied to the piezoelectric element.

In an operation 408, a pharmaceutical or drug located within the disposable portion of the inhaler may aerosolize via vibrations from the transducer. In some embodiment, the medicament dose may be a single pre-metered dose pellet of a dry powder drug composition comprising at least one medicament. The pellet may be formed by compressing a dry powder drug composition. As used herein, the pre-metered dose pellet may include a container that is suitable for containing a dose of dry powder medicament. In some embodiment, the dose pellet may be arranged within the disposable portion of inhaler, which comprises a base sheet in which pre-metered dose pellet is formed to define pockets therein for containing distinct medicament doses and a dose chamber which is sealed in such a manner that the seal of the dose chamber can be peeled there by providing access to the medicament of the pre-metered dose pellet.

In an operation 410, the aerosolized pharmaceutical or drug may be delivered to the user through a dosing chamber and mouthpiece located within the disposable portion of the inhaler. In some embodiment, inhaler may be configured to activate the transducer one or more times to deliver a complete pharmaceutical dose from a dose pellet and dose chamber to a user.

Although the present embodiments have been described in detail for the purpose of illustration based on what is currently considered to be the most practical and preferred embodiments, it is to be understood that such detail is solely for that purpose and that the embodiments are not limited to the disclosed preferred features, but, on the contrary, is intended to cover modifications and equivalent arrangements that are within the scope of the appended claims. For example, it is to be understood that the features disclosed herein contemplate that, to the extent possible, one or more features of any embodiment can be combined with one or more features of any other embodiment.

What is claimed is:

1. A dry powder inhaler, the dry powder inhaler comprising:
a first portion, the first portion including:
a dry powder medicament;
a dosing chamber configured to receive the medicament; and
a mouthpiece configured to deliver the medicament in aerosolized form to the user;
a second portion, the second portion including:
a transducer configured to aerosolize the medicament when the transducer is activated; and
a controller configured to activate the transducer in response to an activation event;
wherein the first portion and the second portion may be coupled together at a connection point.

2. The inhaler of claim 1, wherein the first portion and the second portion include outer tubular housings extending in a longitudinal direction.

3. The inhaler of claim 2, wherein the inhaler is about 5-15 millimeters in diameter and about 80-150 millimeters in length when the first portion and the second portion are coupled together.

4. The inhaler of claim 1, wherein the transducer is configured to vibrate to aerosolize and transfer medicament into the dosing chamber.

5. The inhaler of claim 4, wherein aerosolized medicament is expelled into an air flow conduit of the second portion and is entrained in the user's inhaled air.

6. The inhaler of claim 1, wherein the activation event includes at least one of removal of a seal from the dosing chamber, detection of a user inhalation, or a manual input by a user.

7. The inhaler of claim 1, wherein the second portion further includes a user interface indicating proper function of the inhaler during use.

8. The inhaler of claim 1, the dose chamber is designed in such a manner that it is unsealed there by providing access to the medicament of the dose pellet.

9. The inhaler of claim 1, wherein the first portion is disposable and the second portion is reusable.

10. A method for delivering a dose of a drug with an inhaler, the method comprising:
coupling a first and second portion of the inhaler;
providing a dry powder medicament located in the first portion of the inhaler;
aerosolizing the dry powder medicament via a transducer in the second portion of the inhaler; wherein the transducer is activating in response to an activation event via controller in the second portion of the inhaler;
receiving an aerosolized form of the dry powder in a dosing chamber within the first portion of the inhaler;
delivering the aerosolized dry powder through a mouthpiece of the first portion of the inhaler.

11. The method of claim 10, wherein the first portion and the second portion include outer tubular housings extending in a longitudinal direction.

12. The method of claim 11, wherein the inhaler is about 5-15 millimeters in diameter and about 80-150 millimeters in length when the first portion and the second portion are coupled together.

13. The method of claim 10, wherein the transducer is configured to vibrate to aerosolize and transfer medicament into the dosing chamber.

14. The method of claim 13, wherein aerosolized medicament is expelled into an air flow conduit of the second portion and is entrained in the user's inhaled air.

15. The method of claim 10, wherein the activation event includes at least one of removal of a seal from the dosing chamber, detection of a user inhalation, or a manual input by a user.

16. The method of claim 10, further including:
indicting proper function of the inhaler during use via a user interface within the second portion of the inhaler.

17. The method of claim 10, wherein the dose chamber is designed in such a manner that it is unsealed there by providing access to the medicament of the dose pellet.

18. The method of claim 10, wherein the first portion is disposable and the second portion is reusable.
